(19) United States
(12) Patent Application Publication
(10) Pub. No.: US 2025/0210130 A1
(43) Pub. Date: Jun. 26, 2025

(54) DIFFUSION-BASED GENERATIVE AI METHODS FOR PROTEIN AND DRUG DESIGN

(71) Applicant: Stephen Gbejule Odaibo, Sugar Land, TX (US)

(72) Inventor: Stephen Gbejule Odaibo, Sugar Land, TX (US)

(73) Assignee: Deep EigenMatics LLC, Sugar Land, TX (US)

(21) Appl. No.: 19/077,065

(22) Filed: Mar. 12, 2025

(51) Int. Cl. G16B 15/30; G16B 40/20

(52) U.S. Cl. CPC G16B 15/30; G16B 40/20

(57) ABSTRACT

Methods and apparatus for determining protein and ligand structure, for identifying ligand docking sites, and for obtaining both peptide and non-peptide drug ligand candidates for target proteins are presented. Methods include receiving a plurality of protein-ligand complex structures at a processor, converting to volumetric probability representation, and generating a training dataset by sequentially transforming the voxel-wise probability distributions. A discrepancy measure between consecutive transformations is bounded; that discrepancy measure between each state and the final diffused state progressively decreases; and localization probability of each residue summed over the diffusion volume is constant. A neural network is trained to learn protein and ligand residue localization, given a diffused representation. The methods serve to generate a protein structure given its sequence; or to generate a candidate ligand structure for a given target protein, given only ligand residue composition; or to determine promising candidate peptide and non-peptide drug ligands for synthesis.

### FIELD OF THE INVENTION

The present invention relates generally to Artificial Intelligence (AI) and Machine Learning (ML) methods for protein and drug ligand design and structure determination, and specifically to use of diffusion-based generative models for protein and drug ligand design and structure determination.

### BACKGROUND OF THE INVENTION

The research and development pipeline for new drugs is tremendously expensive and lengthy, often costing over $2 billion and more than 10 years to get a single candidate drug through clinical testing phases. Yet despite the exorbitant investment of time and resources, a high percentage of drugs fail in the clinical testing phases. Generative AI methods such as diffusion-based models have great potential to facilitate the development of new effective drugs. However, one obstacle is that diffusion models were not originally designed to solve problems in protein and drug structure and design. Furthermore, in its standard form, diffusion models are not suited for these types of problems.

While there have been a number of efforts applying diffusion-based models towards protein structure and design problems, there remains a significantly unmet need for an approach that takes into account the dynamics, precision, and fine structure of proteins, protein interactions, and protein-mediated cellular signaling. In particular, proteins are both dynamic and precise, whereby ostensibly similar structural conformations can sometimes encode markedly different functions. In particular, a well-known maxim in machine learning is that a model is only as good as the data on which it was trained. Diffusion models involve first generating training data by sequentially destructuring a data source, and then training a neural network on the generated training data; with the aim of getting the neural network to sequentially recover the original data. As such, when the objective is protein and drug design, it is critical that the training data generation aspect is specifically tailored to effectively capture the fine structure and dynamics of the proteins and protein interactions.

Diffusion models were first introduced in 2015 (Sohl-Dickstein, Jascha, et al. “Deep Unsupervised Learning Using Nonequilibrium Thermodynamics.” International Conference on Machine Learning. PMLR, 2015). In its standard form, the idea is that a neural network can learn to recover the original structure of a data instance from a sequence of randomly perturbed instances which progress from fully structured to fully diffused, provided the sequence changes slowly enough between one state and the next. The state change generally involves randomly sampling from a gaussian, and therefore the final diffused state generally approaches an isotropic gaussian—i.e. white noise with no recognizable structure remaining.

As noted, there have been a number of efforts to apply this idea to the field of protein design and protein structure determination. But most of the efforts have ported the original form of diffusion models, by relying on gaussian noising transition process (and subsequent neural network denoising) of structure parameters, as well as random sampling in generation. However, the original diffusion model approach of gaussian noising and randomness is not suited for the very special purpose of biologically-relevant protein design and structure determination. The standard diffusion model approach neither caters to the most critical aspects of protein structural dynamics nor the precision of protein-ligand interactions.

Firstly, changes in the conformational structure of proteins have biological significance, for instance one conformational structure may switch-off a downstream signaling pathway, while another grossly similar conformational structure of that protein may switch off that same signaling pathway. Secondly, the precise location of the amino acid residues of proteins is often both uncertain and changing with time. Thirdly, in order to attain an effective machine learnable signal, there is a need for the diffusion process to be slowly changing in a manner that is controlled and trackable.

Together, these requirements highlight an important unmet need in the application of diffusion-based models towards biologically-relevant protein and drug design and structure determination. We address this unmet need by presenting the invention disclosed herein. One aspect of the invention includes a Discrepancy Guided and Gated Volumetric Probability Diffusion (DG-VPD) process wherein, we use a discrepancy measure to gate a voxel-wise probability distribution between sequential destructuring steps; and we use the same discrepancy measure to guide the process towards the diffused state.

By addressing the aforementioned important unmet needs, the methods disclosed in this invention present a diffusion-based process tailored towards biologically-relevant protein design and structure determination, thereby increasing the likelihood of success of discovering and manufacturing novel and effective drugs and therapies.

### OBJECTS OF THE INVENTION

It is an object of this invention to provide a system, method, and apparatus for protein and ligand design and structure determination using diffusion-based models; wherein the destructuring process is discrepancy guided and gated, and wherein the destructuring process avoids random gaussian noising.

Another object of this invention is to provide a system, method, and apparatus for protein structure determination, wherein given an amino acid sequence, the method yields the corresponding protein structure.

Another object of this invention is to provide a system, method, and apparatus for peptide ligand structure and docking site determination, given ligand amino acid sequence and target protein sequence and structure.

Yet another object of the invention is to provide a system, method, and apparatus for determining the docking site of a non-peptide ligand, given the target protein sequence and structure, and given a representation of the non-peptide ligand.

Yet another object of the invention is to provide a system, method, and apparatus for generating a candidate peptide ligand, given a target protein sequence and structure and only an amino acid composition of the ligand.

Yet other objects, advantages, and applications of the invention will be apparent from the specifications and drawings included herein.

### SUMMARY OF THE INVENTION

The invention disclosed herein includes a method comprising receiving a plurality of protein sequences and their corresponding structure representations. This is the training data source, from which the training data will subsequently be generated via the Discrepancy Guided and Gated Volumetric Probability Diffusion (DG-VPD) process described further below.

The plurality of protein structure representations in the training data source are then transformed into a volumetric probability representation, wherein the structure is represented within a three-dimensional grid consisting of voxel units. For each voxel, there is associated a probability distribution over the amino acid constituents of the protein, and also over a null element to indicate the probability of the voxel being empty. The probability distribution indicates the probability of any given amino acid residue being present in the given voxel.

Discrepancy Guided and Gated Volumetric Probability Diffusion (DG-VPD) is then applied to a subject for diffusion, which by way of example and not limitation, could be a protein, peptide drug ligand, or non-peptide drug ligand. DG-VPD has an associated discrepancy measure which by way of example and not limitation could be a Kullback-Leibler divergence, a Jensen-Shannon divergence, Wasserstein distance, f-divergence, or cross-entropy, amongst others. For each voxel, each step of the destructuring process involves an update to the associated probability distribution such that the discrepancy measure between the current distribution and the updated distribution is bounded by a hyperparameter function. This is the gating aspect of DG-VPD.

In addition to the discrepancy gating, there is also probability conservation gating in DG-VPD; whereby for any given residue in the diffusion subject, the sum of voxel-wise probabilities across all voxels is always 1 throughout the destructuring process. As a consequence of this, the sum across all voxels of the voxel-wise probability of voxel emptiness is a constant, N−Z, throughout the destructuring process; where N is the number of voxels available for diffusion, and Z is the number of residues in the diffusion subject. Another consequence of this is a probability flux conservation across the surface of any enclosed volume.

Additionally, for the plurality of destructuring iterations, for each voxel, the discrepancy measure between the probability distribution of each state and the final diffused state is lower—in an expectation sense—than the discrepancy measure between the probability distribution of the successive state and the final diffused state. In other words, the destructuring process progressively approaches its goal in a controlled manner measured by the discrepancy measure. This is the discrepancy guidance aspect of DG-VPD.

In one embodiment of the invention, in the final diffused state, the subjects of diffusion are uniformly distributed within the diffusion space. For instance, in the final state there is equal probability of finding any of the amino acid residues in any of the available voxels.

The plurality of states generated by the DG-VPD sequential destructuring process are all stored for use as training data.

A neural network is trained on the generated training data, starting from the final diffused state, the neural network learns to recover the preceding state, ultimately learning to recover an approximation of the original undiffused state.

The trained neural network can then be used for inference.

In some embodiments of the invention, the training data source consists of protein-ligand structures in bound complex, wherein the ligand is a peptide. In such embodiments, the DG-VPD destructuring process proceeds in a manner whereby the peptide ligand is the subject for diffusion. The target protein structure on the other hand serves as the conditional, wherein generative inference will ultimately be done of the ligand given the target protein. Hence, the DG-VPD destructuring process occurs relative to the target protein structure.

Furthermore, in such embodiments, during generative inference, the peptide ligand sequence and target protein sequence and structure are inputs, while the peptide ligand bound state structure and docking site are the output of the method.

In some other embodiment of the invention, the input includes the target protein sequence and structure, and the ligand amino acid composition. However it does not include the ligand sequence. Instead, sequence assignment is learned by another neural network; wherein the training data consist of protein-ligand pairs including ligand residues and their positions, along with their respective associated target proteins' sequences and structures. The training labels are the connections (i.e. sequence info) between the ligand residues.

In this embodiment, a locator neural network generates the locations of the amino acid residues, while a connector neural network generates the connections between residues.

In some other embodiments, the docking site of non-peptide drug ligands are generated given the target protein and non-peptide drug representation.

In summary, the invention disclosed herein consists of systems, methods, and apparatus to use diffusion-based generative models for protein design and structure determination. In particular, a Discrepancy Guided and Gated Volumetric Probability Diffusion (DG-VPG) destructuring process is used to sequentially destructure the training data source, thereby generating a training data set. The generated training data set is used to train a neural network which, given a final diffused representation of a composition of amino acids, can infer their original positions, and therefore their docking site on the target protein. For embodiments whereby the ligand sequence is also given, the sequence information enables output of the ligand structure at inference. Similarly, non-peptide drug ligands can be determined using the methods disclosed herein. The DG-VPD sequential destructuring method addresses an unmet need for biologically-relevant diffusion-based models that cater specifically to the critical aspects of protein design and structure determination. In particular, protein structure is experimentally uncertain and dynamic, and at the same time protein structure and protein-ligand interactions are highly precise-such that similar-appearing structural conformations of a target protein or a ligand may be functionally opposite. The invention disclosed herein addresses the aforementioned unmet needs, thereby increasing the likelihood that these methods will yield novel and effective drug discovery and synthesis.

The invention consists of several outlined processes below, and their relation to each other, as well as all modifications which leave the spirit of the invention invariant. The scope of the invention is outlined in the claims section.

### BRIEF DESCRIPTION OF THE DRAWINGS

In the following detailed description of the invention, we reference the herein listed drawings and their associated descriptions, in which:

FIG. 1 is an illustration of a protein structure with voxel-wise amino acid residue probability representation (volumetric probability representation).

FIG. 2 is an illustration of a protein structure with voxel-wise amino acid residue probability representation (volumetric probability representation).

FIG. 3 is an illustration of initial and final states of Discrepancy Guided and Gated Volumetric Probability Diffusion (DG-VPD).

FIG. 4 is a four state illustrative example of DG-VPD.

FIG. 5 is an illustrative example of the discrepancy gated aspect of DG-VPD.

FIG. 6 is an illustrative example of the discrepancy guided aspect of DG-VPD and of probability conservation gating.

FIG. 7 is an illustrative example of a target protein and a peptide ligand.

FIG. 8 is an illustrative example of initial [t=0] and final [t=T] states of DG-VPD on a peptide ligand conditioned on target protein structure.

FIG. 9 is an illustration of a four state example of DG-VPD on a peptide ligand conditioned on target protein structure.

FIG. 10 is an illustration of a four state example of DG-VPD on a non-peptide drug ligand conditioned on target protein structure.

FIG. 11 is an exemplary schematic of a protein structure determination embodiment.

FIG. 12 is an exemplary schematic of an embodiment of the invention for determining the docking site of a non-peptide drug ligand, given the non-peptide drug ligand identity and a target protein structure.

FIG. 13 is an exemplary schematic of a peptide ligand bound state structure and docking site determination embodiment.

FIG. 14 is an exemplary schematic of an embodiment for generating a peptide ligand structure and docking site, given target protein structure and ligand amino acid composition only.

FIG. 15 is an example of a computing environment.

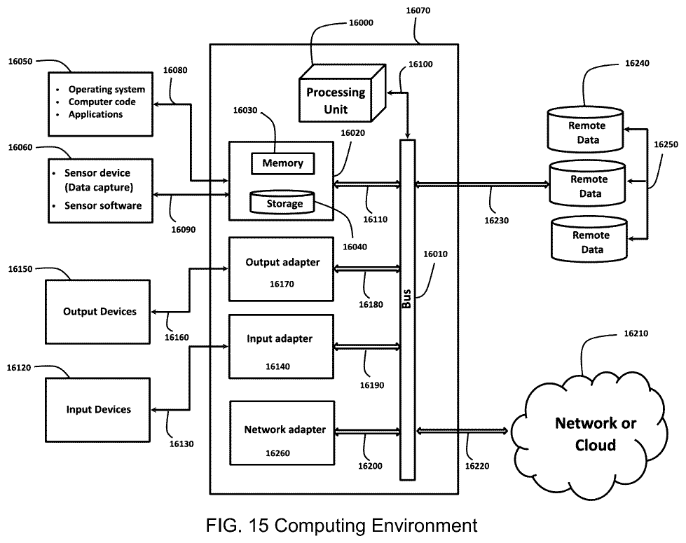

### DETAILED DESCRIPTION OF THE PREFERRED EMBODIMENTS

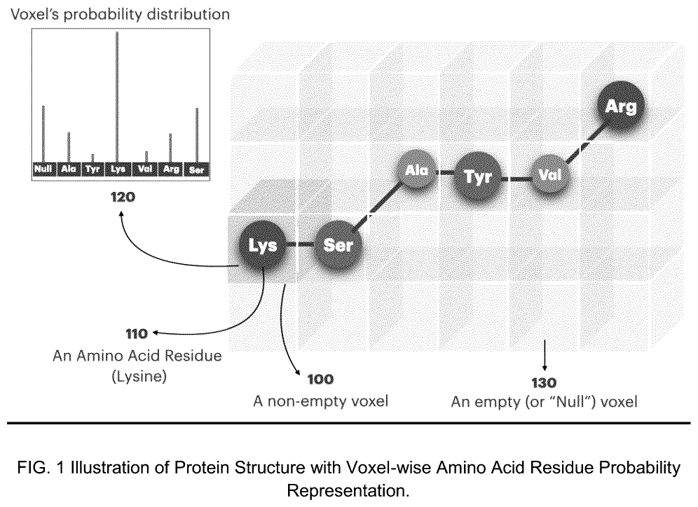

The illustration in FIG. 1 is a preferred embodiment of a volumetric probability representation of a protein structure. In this example, the first amino acid residue 110 is lysine and it is contained primarily in the non-empty voxel 100. The voxel's associated probability distribution 120 is illustrated and the domain consists of the protein's constituent amino acids {Lys, Ser, Ala, Tyr, Val, Arg} and {Null} for empty. As expected, the probability that the voxel holds lysine is higher than the probability that it is empty or that it holds any other of the constituent amino acids.

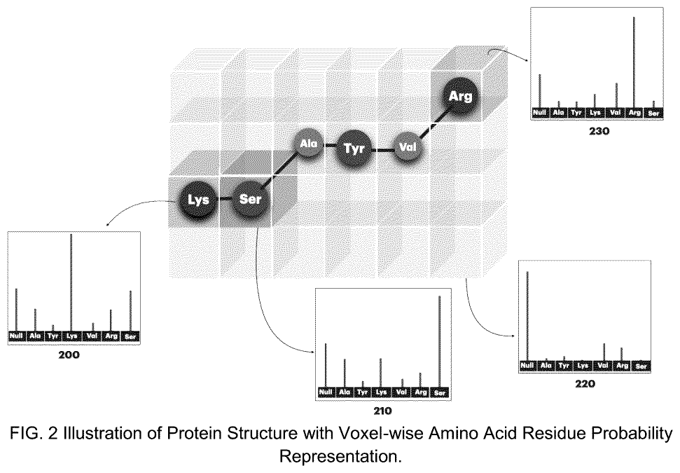

FIG. 2 illustrates the same protein as FIG. 1, here, in addition to the probability distribution of the primary voxel of the lysine residue 200, also depicted are the probability distributions for the primary voxel of the serine residue 210, the primary voxel of the arginine residue 230, and a primarily empty voxel 220. The neighborhood information is reflected for instance in the primarily serine voxel 210 which has a significant probability of being empty or of instead containing the lysine residue.

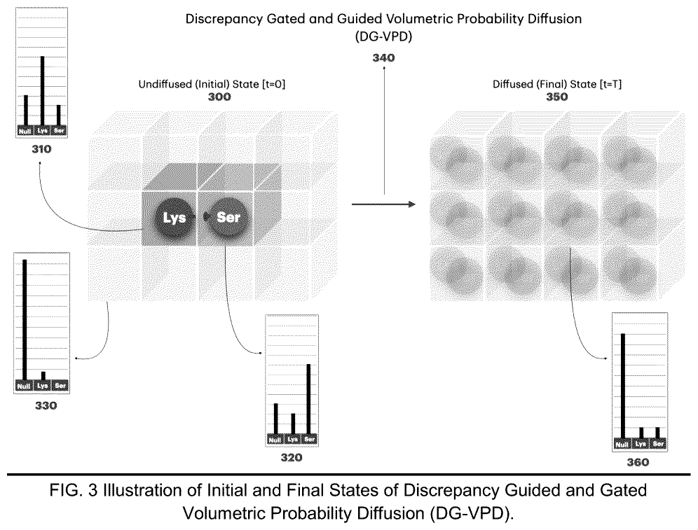

FIG. 3 is an illustrative example of the Discrepancy Guided and Gated Volumetric Probability Diffusion (DG-VPD) destructuring process 340. The process starts with the undiffused (initial) state [t=0] 300 and ultimately yields a diffused (final) state [t=T] 350. In this example, the initial state contains two amino acid residues, lysine and serine, whose primary voxel probability distributions 310 and 320 are shown. The initial state probability distribution 330 of one of the primarily empty voxels is also shown. In the final state 350, in this illustrative example, the probability of finding either of the two amino acids in any given voxel is the same. In other words, each amino acid is uniformly distributed across the diffusion space.

In this embodiment of the invention, in the final diffused state, for any given amino acid subject to the DG-VPD destructuring process, the probability over voxels available for diffusion is uniform.

Furthermore, in one embodiment, the DG-VPD destructuring process respects a probability conservation constraint, thereby including probability conservation gating in its gating mechanism. In particular, in this embodiment, a per residue view gate is enforced as follows:

Σᵢ₌₁^{N}Pᵢ^{[t]}(αⱼ)=1 for all t and for all j≠0 (i.e. for all amino acid residues),

Σᵢ₌₁^{N}Pᵢ^{[t]}(α₀)=Φ=N−M for all t
where N is the number of voxels available for diffusion, itself a tunable hyperparameter; Pᵢ^{[t]}(x) is the probability of finding residue x in the ith voxel at state t; αⱼ is the jth amino acid residue; do is a ‘null’ residue or emptiness; M is the total number of amino acids in the diffusion subject; and tis the DG-VPD destructuring process iteration count (i.e. state) which starts at t=0 and ends at t=T.

Of note, since there can be multiples of a given amino acid in a protein, it follows that it may occur that the amino acid name of a; and the amino acid name of ax are the same, while j≠k. For instance, there can be multiple lysine residues in a single polypeptide chain, each of the lysine residues however is distinct from the others. In other words, if j≠k then αⱼ≠αₖ.

The above probability gating mechanism is expressed in a per residue view. Similarly, this gating mechanism can be expressed in a per voxel view gate whereby the DG-VPD destructuring process includes a probability conservation gating as follows,

Σⱼ₌₀^{Z}Pᵢ^{[t]}(αⱼ)=1 for all t and for all i
where Z is the number of amino acid residues in the diffusion subject; Pᵢ^{[t]}(x) is the probability of finding residue x in the ith voxel at state t; and αⱼ (for j≠0) is the jth amino acid residue in the sequence; α₀ represents emptiness (or ‘null’ residue); and tis the DG-VPD destructuring process iteration count (i.e. state) which starts at t=0 and ends at t=T.

In this embodiment, summation over residues is from 0 to Z, thereby spanning Z+1 items because a voxel can be empty—in other words it can contain emptiness aka a ‘null’ residue. Furthermore, the total number, Φ, of null residues indicates the number of empty voxels at state t=0. In the aforementioned embodiment, this quantity is incorporated into the gating mechanism such that,

Σᵢ₌₁^{N}Pᵢ^{[t]}(‘Null’)=Σᵢ₌₁^{N} Pᵢ^{[t]}(α₀)=Φ=N−Z for all t,
where Z is the total number of non-null residues (i.e. amino acids) in the diffusion subject (e.g. a polypeptide) and N is the total number of voxels that comprise the diffusion space.

Generally, Φ is much greater than 1 since in most instances, the volumetric grid consists of a plurality of primarily empty voxels, i.e. it is sparsely occupied. Therefore typically Φ>>1. However, this is simply by way of example and is in no way a limitation of the invention.

At state t=0 of the DG-VPD destructuring process it holds that given any amino acid residue αⱼ (where j≠0),

Pᵢ^{[0]}(αⱼ)=0 for most voxel index i,

Σ_{i∈S}Pᵢ^{[0]}(αⱼ)=1 where S is a set of indices of voxels in the local neighborhood of the primary voxel of αⱼ.

In this embodiment, for the final diffused state, t=T, the following holds for each amino acid residue,

$${P}_{i}^{[ T ]} ( {α}_{j} ) = \frac{1}{N}$$

for every voxel index i and for every residue index j≠0.

Therefore summing across all voxels available for diffusion, it holds that,

$${\sum }_{i = 1}^{N} \qquad {P}_{i}^{[ T ]} ( {α}_{j} ) = N * ( \frac{1}{N} ) = 1$$

for all j≠0. This is as expected per probability conservation constraint which holds for all t.

Similarly, in this embodiment, the final diffused state, t=T, the following holds regarding the null dispersion,

$${P}_{i}^{[ T ]} ( ' Null ' ) = {P}_{i}^{[ T ]} ( {α}_{0} ) = \frac{Φ}{N}$$

for every voxel index i.

Therefore summing across all voxels available for diffusion, it holds that,

$${\sum }_{i = 1}^{N} \qquad {P}_{i}^{[ T ]} ( ' Null ' ) = {\sum }_{i = 1}^{N} \qquad {P}_{i}^{[ T ]} ( {α}_{0} ) = N * ( \frac{Φ}{N} ) = Φ .$$

The probability conservation gating is enforced for each step of DG-VPD, therefore it follows that for any given enclosed volume, for any given amino acid residue, αⱼ, for any given DG-VPD sequence interval [t=a to t=b], conservation of the probability flux across the enclosure is enforced via the probability gating. In particular,

Σ_{k∈C}Σₜ₌ₐ^{b}ΔPₖ^{[t]}(αⱼ)=−Σ_{k∉C}Σ¿t=a for any a,b such that 0≤a<b<T
where C is a set of indices of voxels within some enclosed volume within the diffusion space, and where,

$$Δ {P}_{k}^{[ t ]} ( {α}_{j} ) = {P}_{k}^{[ t + 1 ]} ( {α}_{j} ) - {P}_{k}^{[ t ]} ( {α}_{j} )$$

$$\begin{matrix}{\sum }_{k ∈ C} & {\sum }_{t = a}^{b} & ( {P}_{k}^{[ t + 1 ]} ( {α}_{j} ) - {P}_{k}^{[ t ]} ( {α}_{j} ) ) = - {\sum }_{k ∉ C} & {\sum }_{t = a}^{b}\end{matrix} ( {P}_{k}^{[ t + 1 ]} ( {α}_{j} ) - {P}_{k}^{[ t ]} ( {α}_{j} ) )$$

In other words, for any given residue, the flux of localization probability density across the surface of the enclosure volume V_{c} is conserved in that any efflux from V_{c} is influx into the exterior of V_{c} and vice versa—any influx into V_{c} is efflux from the exterior of V_{c}.

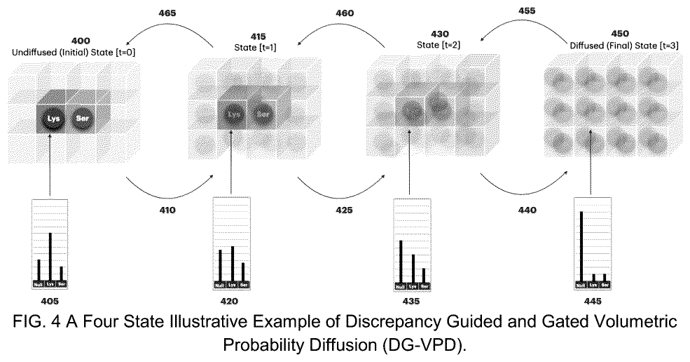

FIG. 4 is an illustrative example of a four state configuration of DG-VPD. The initial undiffused state [t=0] 400 has in it an oligopeptide with two amino acids, lysine and serine. The probability distribution 405 of the primary voxel of the lysine residue shows lysine as the most probable occupant of the voxel, next most likely is that the voxel is empty, and least probable is that it contains the serine residue. The DG-VPD destructuring proceeds from state [t=0] 400 ultimately to final state [t=T] 450. At the final state, the probability of the lysine residue, α₁, being in any given voxel is 1/12; and the same holds for α₂, the serine residue. The 1/12 arises because as earlier mentioned, amino acid residues are always distinct (i.e. n=1), and here there are 12 voxels available for diffusion, i.e. N=12. On the other hand, in the final state [t=T], the probability of any given voxel being empty is 10/12. This is because in this example, ϕ=10 and N=12.

Of note, the state t=T uniform distribution pattern described in the above embodiment is simply one embodiment of the invention and not a limitation. Other final state patterns (i.e. boundary conditions) can be implemented in other embodiments of the invention.

Also in FIG. 4, the two intermediate states are shown, state [t=1] 415 and state [t=2] 430. Furthermore, the probability distribution of the lysine residue's primary voxel is shown across all four states of the example. Through the DG-VPD transformation sequence, the probability distribution of the voxel changes from 405 to 420 to 435 to 445. It changes gradually from its initial form to its final form while being guided by a discrepancy measure, and gated both by a discrepancy measure as well as by probability conservation.

By way of example and not limitation, the discrepancy measure could be a Kullback-Leibler divergence, a Jensen-Shannon divergence, a Wasserstein distance, or a cross-entropy amongst others. One aspect of the gating, discrepancy gating, involves, for each voxel, bounding the discrepancy measure between the current and updated probability distributions. This, in addition to probability conservation gating, confers fine structure control on the destructuring process and enforces a continuity in the form of the distribution. By doing so, DG-VPD finely preserves a continuous machine learnable signal of fine structure, spatial relationships, and biological relevance in the generated training data.

Also in FIG. 4, a residue locator neural network training process is illustrated, wherein preceding states are successively recovered starting from the final diffused [t=3] state 450 and ending in the undiffused [t=0] state 400. Given state [t=3] 450, the neural network learns to predict state [t=2] 430; given state [t=2] 430, it learns to predict state [t=1] 415; and given state [t=1] 415, it learns to predict state [t=0] 400.

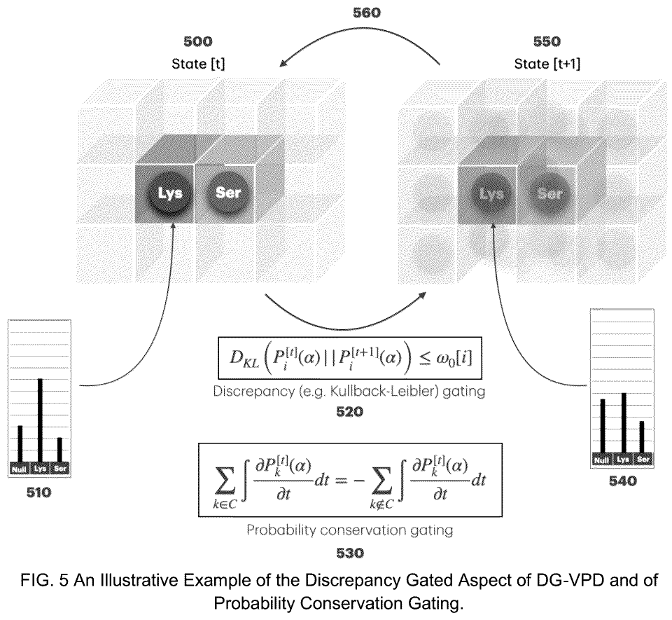

FIG. 5 is an illustration of discrepancy gating between a state [t] 500 and the subsequent state [t+1] 550. In the exemplified embodiment, the discrepancy measure 520 is the Kullback-Leibler divergence, given by,

$${D}_{K L} ( {P}_{i} ‖ {Q}_{i} ) := - {\sum }_{j = 0}^{Z} \qquad {P}_{i} [ j ] log \frac{{Q}_{i} [ j ]}{{P}_{i} [ j ]}$$

The discrepancy gating between states [t] and [t+1] is given by,

D_{KL}(Pᵢ^{[t]}(α)∥Pᵢ^{[t+1]}(α))≤ω₀[i] for all voxel indices i
where ω₀[i] is a tunable hyperparameter function of the system.

One expression of probability conservation gating is probability flux conservation 530, which we described earlier in this disclosure in discrete form. For any given residue, the probability flux across any enclosed volume in the diffusion space conserves the residue.

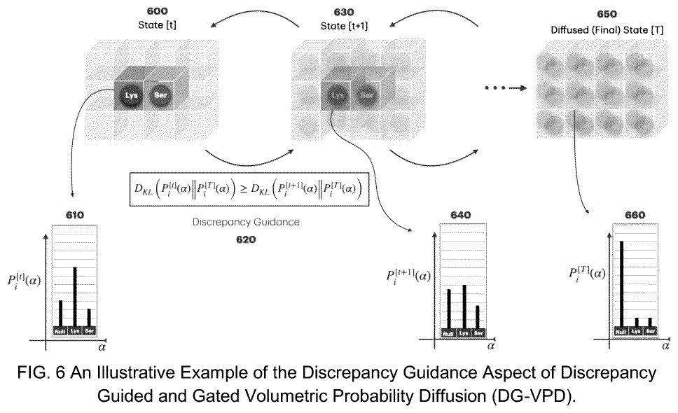

FIG. 6 illustrates the discrepancy guidance aspect of DG-VPD. Discrepancy guidance is the following: in an expectation sense, for each voxel, the associated probability distribution's ‘distance-to-goal’ is to decrease with successive state updates in DG-VPD. Wherein, ‘distance-to-goal,’ is the discrepancy measure between the probability distribution of a given state [t] and the probability distribution of the final diffused state [T] 650. The inequality 620 depicts this guidance objective, exemplified between a state [t] 600 and the subsequent state [t+1] 630. The discrepancy guidance objective is given by,

D_{KL}(Pᵢ^{[t]}(α)∥Pᵢ^{[T]}(α))≥D_{KL}(Pᵢ^{[t+1]}(α)∥Pᵢ^{[T]}(α)) for all i and for all t

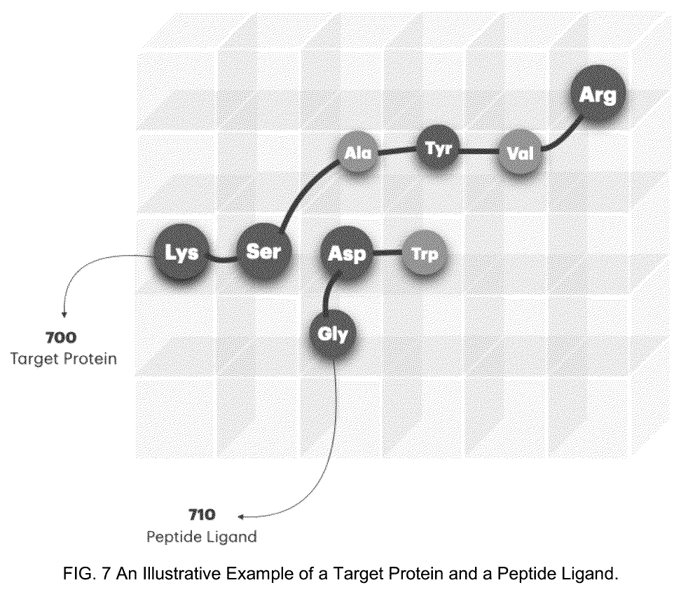

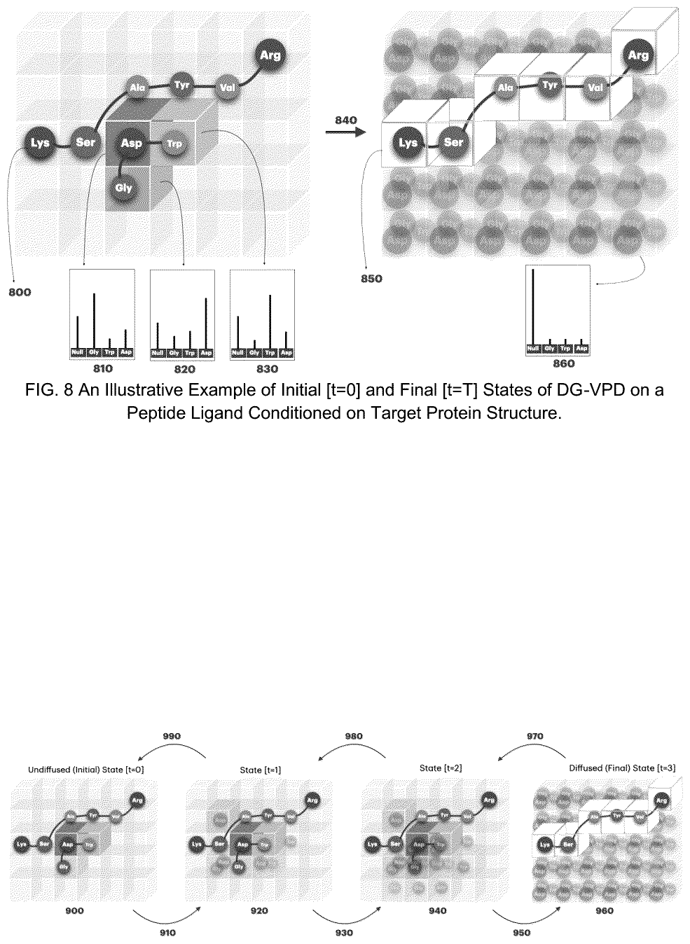

FIG. 7 illustrates a target protein 700 and an associated oligopeptide ligand 710 consisting of three amino acids. Further, FIG. 8 depicts the state [t=0] probability distributions 810, 820, and 830 of each of the three primary voxels of the oligopeptide ligand's three amino acids: aspartic acid, glycine, and tryptophan respectively. The DG-VPD destructuring process 840 ultimately yields the fully diffused state [t=T], wherein the probability distribution 860 associated with each of the voxels is uniform. The target protein itself is the conditional and therefore is not itself a subject of the diffusion, hence in this embodiment, it is unchanged from its initial state [t=0] 800 to its final state [t=T] 850.

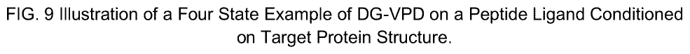

FIG. 9 illustrates a four state example whose initial state 900 and final state [t=3] 960 are also illustrated in FIG. 8. The first DG-VPD destructuring step 910 transforms the initial state 900 to the state [t=1] 920; the second DG-VPD destructuring step 930 transforms state [t=1] 920 to state [t=2] 940; and the third and final DG-VPD destructuring step 950 transforms state [t=2] 940 into the final diffused state [t=3] 960. The residue locator neural network in turn learns to sequentially recover the data in steps 970, then 980, then 990, yielding back its inference of state [t=0] 900.

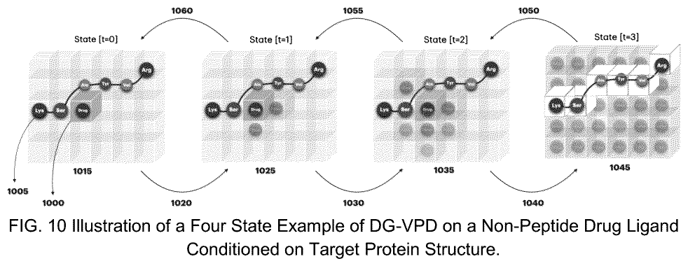

FIG. 10 illustrates the DG-VPD destructuring and subsequent neural network recovery training of a small molecule drug (i.e. non-peptide ligand) 1000 conditioned on the target protein structure 1005. A small molecule drug 1000 is typically of comparable size to a single amino acid residue, hence is encoded in this embodiment as a single node, thereby occupying a single voxel akin to a single residue of a peptide. Small molecule drugs typically have a molecular weight of around 100 to 1000 daltons (grams/mole), with most being less than 500 daltons; while single amino acids have an average molecular weight of 110 daltons. Specifically, the smallest amino acid, glycine, has a molecular weight of 75.07 daltons, while the largest amino acid, tryptophan, has a molecular weight of 204.23 daltons. By comparison, molecular weights of a few small molecule drug examples include the beta-1 adrenergic G-Protein Coupled Receptor (GPCR) blocker, atenolol (266.34 daltons); the beta-2 adrenergic GPCR agonist, albuterol (239.31 daltons); the common analgesic, acetaminophen (151.16 daltons); the common non-steroidal anti-inflammatory drug, Ibuprophen (206.29 daltons); and the common loop diuretic, furosemide (330.75 daltons). By comparison, molecular weights of some common polypeptide ligands include: angiotensin II, an 8 amino acid oligopeptide agonist of the GPCRs AT1R and AT2R, has a molecular weight of 1046.18 daltons; insulin, a 51 amino acid polypeptide agonist of the insulin receptor, has molecular weight of 5808 daltons.

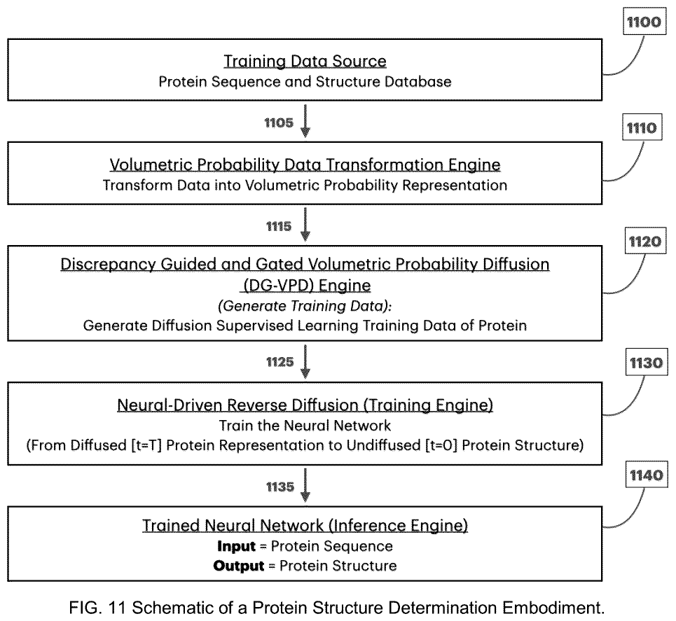

FIG. 11 is a schematic flow chart of a protein structure determination embodiment. In this embodiment, the training data source 1100 consists of sequence and structure representations of a plurality of proteins. This data is transmitted 1105 to a volumetric probability data transformation engine 1110, which transforms the data into a volumetric probability representation; wherein the protein is represented within a three dimensional grid, whereby with each voxel of the grid is associated a probability distribution indicating the probability of any given amino acid residue being located within that voxel.

The volumetric probability representation of the dataset is then transmitted 1115 from the volumetric probability data transformation engine 1110 to the Discrepancy Guided and Gated Volumetric Probability Diffusion (DG-VPD) engine 1120, which generates the training data as earlier described in FIGS. 3-10. In particular, the DG-VPD engine 1120 generates diffusion supervised training data from each of a diverse plurality of protein structure representations obtained from the volumetric probability data transformation engine 1110.

To reiterate, in one embodiment of the invention, the equations governing the DG-VPD process are as follows:

An Embodiment of Discrepancy Guided and Gated Volumetric Probability
Diffusion (DG-VPD) Equations

| | | |
|---|---|---|
| (1) | ${\sum }_{i = 1}^{N} {P}_{i}^{[ t ]} ( {α}_{j} ) = 1$ | for all t and for all j ≠ 0 |
| (2) | ${\sum }_{j = 0}^{Z} {P}_{i}^{[ t ]} ( {α}_{j} ) = 1$ | for all t and for all i |
| (3) | Σᵢ₌₁^{N} Pᵢ^{[t]}(α₀) = Φ = (N − Z) | for all t |
| (4) | ${P}_{i}^{[ T ]} ( {α}_{j} ) = \frac{1}{N}$ | for all i and for all j ≠ 0 |
| (5) | ${P}_{i}^{[ T ]} ( {α}_{0} ) = \frac{Φ}{N} = \frac{( N - Z )}{N}$ | for all i |
| (6) | ${\sum }_{k ∈ C} {\sum }_{t = a}^{b} Δ {P}_{k}^{[ t ]} ( {α}_{j} ) = - {\sum }_{k ∉ C} {\sum }_{t = a}^{b} Δ {P}_{k}^{[ t ]} ( {α}_{j} )$ | for any a, b: 0 ≤ a < b < T and for all j |
| (7) | D_{KL} (Pᵢ^{[t]}(α) \|\| Pᵢ^{[t+1]}(α)) ≤ ω₀[i] | for all i |
| (8) | D_{KL} (Pᵢ^{[t]}(α) \|\| Pᵢ^{[T]}(α)) ≥ | for all t and for all i |
| | D_{KL} (Pᵢ^{[t+1]}(α) \|\| Pᵢ^{[T]}(α)) | |

where N is the number of voxels in the diffusion space; t is the state index and runs from 0 to T; j is the amino acid residue index such that αⱼ (for j≠0) is the jth amino acid; α₀ represents emptiness of a voxel i.e. a ‘null residue’; Z is the number of amino acid residues in the protein; Φ indicates the number of empty voxels in the diffusion space at t=0; C is a set of indices of the voxels within any enclosed volume V_{c} within the diffusion space; D_{KL} is the Kullback-Leibler divergence; and ω₀[i] is a hyperparameter function.

Various embodiments of DG-VPD 1120 can be implemented in terms of how to sequentially destructure the data in a manner guided by and in compliance with the above equations. In other words, there are a plurality of options for a DG-VPD solver including, but in no way limited to: genetic algorithm; monte-carlo tree search (MCTS); gradient-based solver e.g. stochastic gradient descent, conjugate gradient method; simulated annealing; particle swarm optimization; penalty methods; and dynamic programming amongst others.

While a DG-VPD solver component of the DG-VPD engine 1120 for the purpose of generating high quality training data is generally of higher computational cost than simply randomly sampling from a gaussian to add noise to the data, the precision of the DG-VPD destructuring process is tailored to protein and drug design, and therefore promises higher fidelity fine structure preservation and biological-relevance. This significantly increases the likelihood of yielding novel and effective drugs and therapies over the standard diffusion model approach.

This is essential given the critical importance and exceeding high cost of drug discovery and development, often costing over $2 Billion and 10 years of dedicated research and development to get a single drug through clinical testing phases. Nonetheless, a high percentage of drugs fail in the testing phases despite the extraordinarily high investment. Furthermore, for any machine learning model, the quality and usefulness of results are a direct function of the quality of the training data. Therefore, DG-VPD addresses the critical need to generate high quality training data for protein and drug structure and design.

The training data generated via the DG-VPD engine 1120 is transmitted 1125 to a Neural-Driven Reverse Diffusion training engine 1130, which includes a residue locator neural network that trains on the generated training data via supervised learning. In particular, it starts at the fully diffused state [t=T] and sequentially recovers the preceding state, ultimately recovering a reconstructed version of the origin state [t=0].

The trained locator neural network is outputted 1135 from the training engine 1130 into the inference engine 1140 of which it is a main component. The residue locator neural network is configured to accept a protein sequence as input and to output the protein structure. In particular, given a protein sequence, the protein can be represented in the fully diffused state [t=T] form (e.g. 450 in FIG. 4 or 650 in FIG. 6), from which the trained locator neural network infers the protein structure.

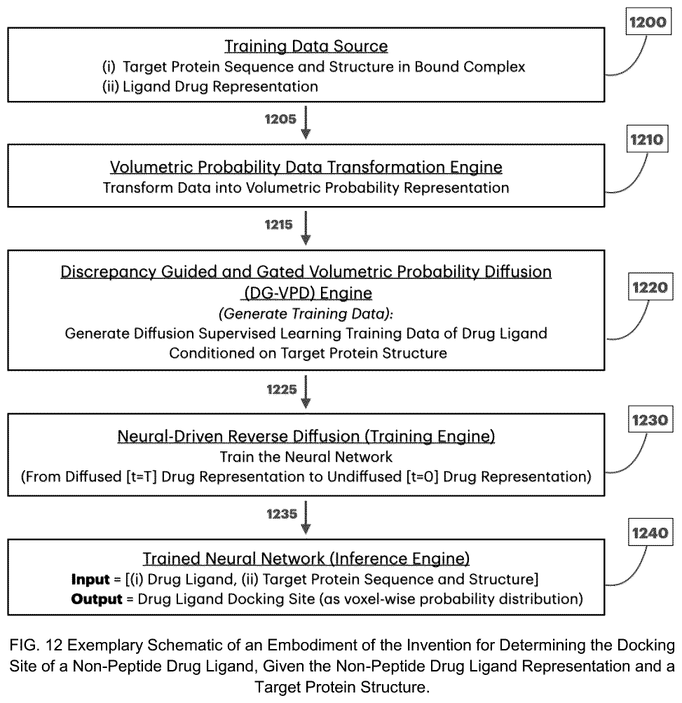

FIG. 12 depicts a schematic of an embodiment of the invention for determining the docking site of a non-peptide drug ligand, i.e. small molecule drug, given a representation of the drug and given a target protein structure. In this embodiment, the training data source 1200 consists of sequence and structure representations of a diverse plurality of target protein bound complexes, wherein the diverse plurality of bound complexes are each of a target protein and a drug ligand. This data is transmitted 1205 to a volumetric probability data transformation engine 1210 which converts it to a volumetric probability representation. Afterwards, the data is transmitted 1215 to the DG-VPD engine 1220 which generates training data. In particular, the DG-VPD engine 1220 generates diffusion supervised learning data from each of a diverse plurality of drug ligand representations conditioned on their respective target protein structures.

The generated training data is then transmitted 1225 to a Neural-Driven Reverse Diffusion training engine 1230, which includes a position locator neural network that trains on the generated training data via supervised learning. In particular, it starts at the fully diffused state [t=T] and sequentially recovers the preceding state, ultimately recovering a reconstructed version of the origin state [t=0]. The trained neural network is outputted 1235 from the training engine 1230 into the inference engine 1240 of which it is a main component. The position locator neural network is configured to accept as input: (i) a non-peptide drug ligand representation and (ii) a target protein sequence and structure. As output, it yields the drug ligand docking site. In particular, given a representation of a non-peptide drug ligand and given a target protein structure, the drug ligand can be represented in the fully diffused state [t=T] form around the target protein (e.g. as depicted in 1045 of in FIG. 10); and then from the fully diffused representation, the trained locator neural network infers the drug ligand's docking site on the target protein.

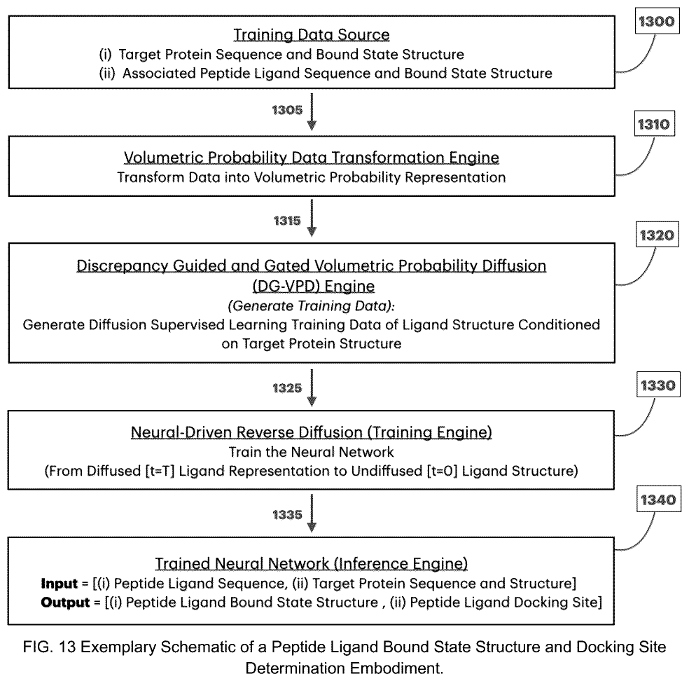

FIG. 13 depicts a schematic flow chart of an embodiment of the invention, wherein the embodiment outputs the bound state structure and docking site of a peptide ligand given the peptide ligand's sequence and a target protein's sequence and structure. The training data source 1300 consists of sequence and structure representations of a diverse plurality of bound complexes, each of a target protein and a peptide ligand. This data is transmitted 1305 to a volumetric probability data transformation engine 1310 which converts it to a volumetric probability representation. Afterwards, the data is transmitted 1315 to a DG-VPD engine 1320 which generates training data. In particular, the DG-VPD engine 1320 generates diffusion supervised learning data from each of a diverse plurality of peptide ligand structures conditioned on their respective target protein structures.

The generated training data is then transmitted 1325 to a Neural-Driven Reverse Diffusion training engine 1330, which includes a residue locator neural network that trains on the generated training data via supervised learning. In particular, it starts at the fully diffused state [t=T] and sequentially recovers the preceding state, ultimately recovering a reconstructed version of the origin state [t=0]. The trained neural network is outputted 1335 from the training engine 1330 into the inference engine 1340 of which it is a main component. The trained residue locator neural network is configured to accept as input: (i) a peptide ligand sequence, and (ii) a target protein sequence and structure. As output, it yields: (i) the peptide ligand bound state structure, and (ii) the peptide ligand docking site. In particular, given a peptide ligand sequence and given a target protein sequence and structure, the peptide ligand can be represented in the fully diffused state [t=T] form around the target protein (e.g. as depicted in 960 of in FIG. 9); and then from the fully diffused representation, the trained residue locator neural network infers the peptide ligand's bound state structure and docking site on the target protein.

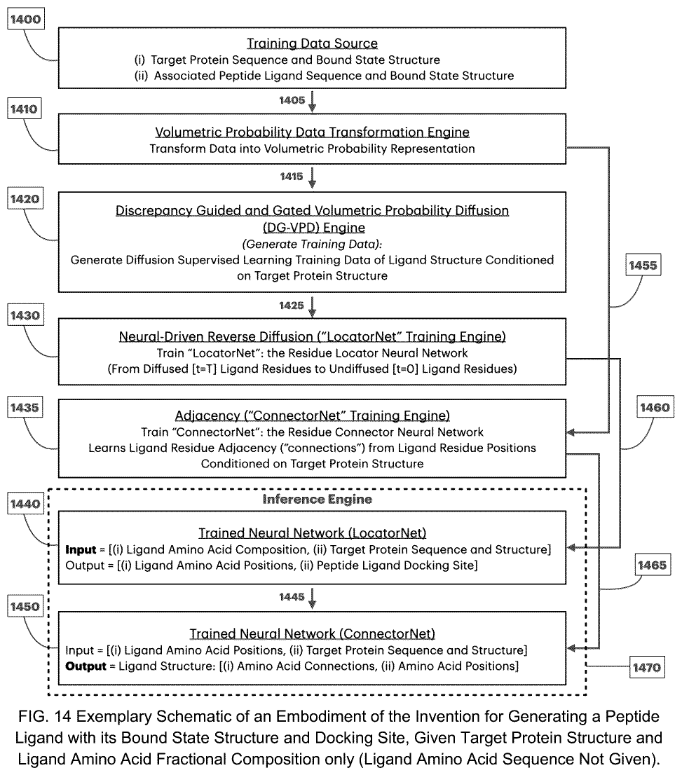

FIG. 14 depicts a schematic flow chart of an embodiment of the invention, wherein the embodiment generates a peptide ligand representation including the bound state structure and docking site of the peptide ligand, given only an amino acid composition (not sequence information), and given the target protein's sequence and structure. The training data source 1400 consists of sequence and structure representations of a diverse plurality of bound state complexes, each of a target protein and a peptide ligand. This data is transmitted 1405 to a volumetric probability data transformation engine 1410 which converts it to a volumetric probability representation. Afterwards, the data is transmitted 1415 to a DG-VPD engine 1420 which generates training data. In particular, the DG-VPD engine 1420 generates diffusion supervised learning data from each of a diverse plurality of peptide ligand structures conditioned on their respective target protein structures.

The generated training data is then transmitted 1425 to a Neural-Driven Reverse Diffusion training engine 1430, which includes a residue locator neural network (“LocatorNet”) that trains on the generated training data via supervised learning. In particular, it starts at the fully diffused state [t=T] and sequentially recovers the preceding state, ultimately recovering a reconstructed version of the origin state [t=0]. The trained neural network is outputted 1460 from the LocatorNet training engine 1430 into the LocatorNet component 1440 of the inference engine 1470. The trained residue locator neural network (LocatorNet) 1440 is configured to accept as input: (i) a peptide ligand amino acid composition, and (ii) a target protein sequence and structure. As output, it yields: (i) the peptide ligand amino acid positions, and (ii) the peptide ligand docking site. In particular, given a peptide ligand amino acid composition (not including sequence), and given a target protein sequence and structure, the peptide ligand can be represented in the fully diffused state [t=T] form around the target protein (e.g. as depicted in 960 of in FIG. 9); and then from the fully diffused representation, the trained LocatorNet neural network infers the peptide ligand's bound state constituent amino acid coordinates and therefore also its docking site on the target protein.

However, to get the peptide ligand's structure, knowing its constituent amino acid positions alone is not sufficient. Its sequence information is also required. For this, an amino acid adjacency neural network training engine (“ConnectorNet” training engine) 1435 receives 1455 training data from the volumetric probability data transformation engine 1410. In particular, the ConnectorNet training engine 1435 learns peptide ligand amino acid adjacency information from peptide ligand amino acid positions conditioned on the target protein structure. The trained ConnectorNet is outputted 1465 from the ConnectorNet training engine 1435 into the ConnectorNet component 1450 of the inference engine 1470. From the locatorNet component 1440 of the inference engine 1470, the connectorNet component 1450 receives 1445 the peptide ligand amino acid positions and the target protein sequence and structure.

The trained residue connector neural network (ConnectorNet) 1450 is configured to accept as input: (i) peptide ligand amino acid positions, and (ii) a target protein sequence and structure. As output, it yields an inference of the peptide ligand's amino acid connections. The peptide ligand structure is then determined by combining the peptide ligand amino acid position information with the sequence information.

Ones with ordinary skill in the art will recognize that the invention disclosed herein can be implemented over an arbitrary range of computing configurations. We will refer to any instantiation of these computing configurations as the computing environment. An illustrative example of a computing environment is depicted in The Computing Environment FIG.

Examples of computing environments include but are not limited to desktop computers, laptop computers, tablet personal computers, mainframes, mobile smart phones, smart television, programmable hand-held devices and consumer products, distributed computing infrastructures over a network, cloud computing environments, or any assembly of computing components such as memory and processing—for example.

As illustrated in The Computing Environment FIG, the invention disclosed herein can be implemented over a system that contains a device or unit for processing the instructions of the invention. This processing unit 16000 can be a single core central processing unit (CPU), multiple core CPU, graphics processing unit (GPU), multiplexed or multiply-connected GPU system, or any other homogeneous or heterogeneous distributed network of processors.

In some embodiment of the invention disclosed herein, the computing environment can contain a memory mechanism to store computer-readable media. By way of example and not limitation, this can include removable or non-removable media, volatile or non-volatile media. By way of example and not limitation, removable media can be in the form of flash memory card, USB drives, compact discs (CD), blu-ray discs, digital versatile disc (DVD) or other removable optical storage forms, floppy discs, magnetic tapes, magnetic cassettes, and external hard disc drives. By way of example but not limitation, non-removable media can be in the form of magnetic drives, random access memory (RAM), read-only memory (ROM) and any other memory media fixed to the computer.

As depicted in The Computing Environment FIG, the computing environment can include a system memory 16030 which can be volatile memory such as random access memory (RAM) and may also include non-volatile memory such as read-only memory (ROM). Additionally, there typically is some mass storage device 16040 associated with the computing environment, which can take the form of hard disc drive (HDD), solid state drive, or CD, CD-ROM, blu-ray disc or other optical media storage device. In some other embodiments of the Invention the system can be connected to remote data 16240.

The computer readable content stored on the various memory devices can include an operating system, computer codes, and other applications 16050. By way of example not limitation, the operating system can be any number of proprietary software such as Microsoft windows, Android, Macintosh operating system, iphone operating system (IOS), or Linux commercial distributions. It can also be open source software such as Linux versions e.g. Ubuntu. In other embodiments of the invention, data processing software and connection instructions to a sensor device 16060 can also be stored on the memory mechanism. The procedural algorithm set forth in the disclosure herein can be stored on—but not limited toαany of the aforementioned memory mechanisms. In particular, computer readable instructions for training and subsequent image classification tasks can be stored on the memory mechanism.

The computing environment typically includes a system bus 16010 through which the various computing components are connected and communicate with each other. The system bus 16010 can consist of a memory bus, an address bus, and a control bus. Furthermore, it can be implemented via a number of architectures including but not limited to Industry Standard Architecture (ISA) bus, Extended ISA (EISA) bus, Universal Serial Bus (USB), microchannel bus, peripheral component interconnect (PCI) bus, PCI-Express bus, Video Electronics Standard Association (VESA) local bus, Small Computer System Interface (SCSI) bus, and Accelerated Graphics Port (AGP) bus. The bus system can take the form of wired or wireless channels, and all components of the computer can be located remote from each other and connected via the bus system. By way of example and not of limitation, the processing unit 16000, memory 16020, input devices 16120, output devices 16150 can all be connected via the bus system. In the representation depicted in The Computing Environment FIG, by way of example not limitation, the processing unit 16000 can be connected to the main system bus 16010 via a bus route connection 16100; the memory 16020 can be connected via a bus route 16110; the output adapter 16170 can be connected via a bus route 16180; the input adapter 16140 can be connected via a bus route 16190; the network adapter 16260 can be connected via a bus route 16200; the remote data store 16240 can be connected via a bus route 16230; and the cloud infrastructure can be connected to the main system bus vis a bus route 16220.

In some embodiment of the invention disclosed herein, The Computing Environment FIG illustrates that instructions and commands can be input by the user using any number of input devices 16120. The input device 16120 can be connected to an input adapter 16140 via an interface 16130 and/or via coupling to a tributary of the bus system 16010. Examples of input devices 16120 include but are by no means limited to keyboards, mouse devices, stylus pens, touchscreen mechanisms and other tactile systems, microphones, joysticks, infrared (IR) remote control systems, optical perception systems, body suits and other motion detectors. In addition to the bus system 16010, examples of interfaces through which the input device 16120 can be connected include but are by no means limited to USB ports, IR interface, IEEE 802.15.1 short wavelength UHF radio wave system (bluetooth), parallel ports, game ports, and IEEE 1394 serial ports such as FireWire, i.LINK, and Lynx.

In some embodiment of the invention disclosed herein, The Computing Environment FIG illustrates that output data, instructions, and other media can be output via any number of output devices 16150. The output device 16150 can be connected to an output adapter 16170 via an interface 16160 and/or via coupling to a tributary of the bus system 16010. Examples of output devices 16150 include but are by no means limited to computer monitors, printers, speakers, vibration systems, and direct write of computer-readable instructions to memory devices and mechanisms. Such memory devices and mechanisms can include by way of example and not limitation, removable or non-removable media, volatile or non-volatile media. By way of example and not limitation, removable media can be in the form of flash memory card, USB drives, compact discs (CD), blu-ray discs, digital versatile disc (DVD) or other removable optical storage forms, floppy discs, magnetic tapes, magnetic cassettes, and external hard disc drives. By way of example but not limitation, non-removable media can be in the form of magnetic drives, random access memory (RAM), read-only memory (ROM) and any other memory media fixed to the computer. In addition to the bus system 16010, examples of interfaces through which the output device 16150 can be connected include but are by no means limited to USB ports, IR interface, IEEE 802.15.1 short wavelength UHF radio wave system (bluetooth), parallel ports, game ports, and IEEE 1394 serial ports such as FireWire, i.LINK, and Lynx.

In some embodiment of the invention disclosed herein some of the computing components can be located remotely and connected to via a wired or wireless network. By way of example and not limitation, The Computing Environment FIG shows a cloud 16210 and a remote data source 16240 connected to the main system bus 16010 via bus routes 16220 and 16230 respectively. The cloud computing infrastructure 16210 can itself contain any number of computing components or a complete computing environment in the form of a virtual machine (VM). The remote data source 16240 can be connected via a network to any number of external sources such as NMR spectrometry devices, X-ray diffraction devices, electron microscopes, imaging devices, imaging systems, or imaging software.

In some embodiment of the invention disclosed herein, a sensor system 16060 which captures and pre-processes data is attached directly to the system. For example, this may be an electron microscope (and associated image processing software); it may be a camera in the case of an imaging system, say for processing distance map photographs; or it may be an X-ray crystallography machine or an NMR spectrometer (and associated software), excetera. Stored in the memory mechanism—16020, 16240, or 16210—are machine learning models, algorithms, and data products developed according to the procedures set-forth herein. Computer-readable instructions are also stored in the memory mechanism, so that upon command, protein structure representation data, its substrates and associated data can be captured or can be received over a network from a remote or local previously collated database. This transmission of data can be done over a wired or wireless network as previously detailed, as the source and/or recipient of the data output can be at a remote location.

The objects set forth in the preceding are presented in an illustrative manner for reason of efficiency. It is hereby noted that the above disclosed methods and systems can be implemented in manners such that modifications are made to the particular illustration presented above, while yet the spirit and scope of the invention is retained. The interpretation of the above disclosure is to contain such modifications, and is not to be limited to the particular illustrative examples and associated drawings set-forth herein.

Furthermore, by intention, the following claims encompass all of the general and specific attributes of the invention described herein; and encompass all possible expressions of the scope of the invention, which can be interpreted—as pertaining to language—as falling between the aforementioned general and specific ends.

What is claimed:

1. A method, comprising:
a. receiving, at a processor, representations of a plurality of proteins, each consisting of:
i. an amino acid sequence, and
ii. an associated structure representation;
b. for each protein, representing, via the processor, the protein's structure as voxel coordinates of its constituent amino acids within a three dimensional grid:
i. wherein with each voxel is associated a probability distribution,
ii. wherein the probability distribution indicates the probability of finding any given constituent amino acid in that voxel;
c. sequentially transforming, via the processor, each voxel's probability distribution such that:
i. a measure of discrepancy between consecutive probability distributions is bounded by a hyperparameter function,
ii. a probability conservation constraint is respected, wherein for any given amino acid residue, the sum of the probabilities across voxels is constant,
iii. the sequential transformation proceeds in a manner to decrease the discrepancy measure between the current state's probability distribution and the final (diffused) state's probability distribution,
iv. the resulting sequence of probability distributions arising from the above transformations—the generated training data—are stored in the processor's associated memory;
d. training a neural network, via the processor, to start at the final diffused state, and sequentially recover the preceding states, thereby ultimately yielding an approximation of the origin state:
i. wherein the training dataset is the generated training data,
ii. wherein for each voxel, the training loss includes a measure of discrepancy between the neural network's approximation of each state's probability distribution and the state's probability distribution in the generated training data,
iii. wherein the neural network is configured to accept the amino acid sequence of a protein as input, and to output the structure of the corresponding protein;
e. using the trained neural network to obtain the structure of a protein, given the protein's amino acid sequence.

2. The method of claim 1, wherein the final (diffused) state is such that for each voxel, the associated probability distribution is uniform over the amino acid residues, wherein each amino acid residue of the protein has equal probability of being in any given voxel.

3. The method of claim 2, wherein the discrepancy measure is a Kullback-Leibler divergence.

4. The method of claim 2, wherein the discrepancy measure is a Jensen-Shannon divergence.

5. A method, as in the method of claim 1, for determining the structure of a protein given its amino acid sequence, wherein the method is also for diagnostically detecting a proteinopathy, the method further comprising:
a. using the method of claim 1, to determine a structure of a protein given its amino acid sequence;
b. comparing the predicted structure to the experimentally determined structure of that protein taken from a sample in a human, animal, or plant.

6. A method, comprising:
a. receiving, at a processor, representations of a plurality of protein-ligand complexes, wherein each ligand is represented as a linear graph consisting of:
i. a sequence of nodes, and
ii. an associated structure representation,
and wherein each target protein is represented as:
i. an amino acid sequence, and
ii. an associated structure representation;
b. for each target protein and each associated ligand, representing, via the processor:
i. the ligand's structure as voxel coordinates of its constituent nodes within a three dimensional grid, wherein with each voxel is associated a probability distribution which indicates the probability of finding any given constituent node in that voxel,
ii. the target protein's structure as voxel coordinates of its constituent amino acids within a three dimensional grid, wherein with each voxel is associated a probability distribution which indicates the probability of finding any given constituent amino acid in that voxel;
c. for each ligand, sequentially transforming each voxel's probability distribution over the ligand's constituent nodes, such that:
i. a measure of discrepancy between consecutive probability distributions is bounded by a hyperparameter function,
ii. a probability conservation constraint is respected, wherein for any given amino acid residue, the sum of the probabilities across voxels is constant,
iii. the sequential transformation proceeds in a manner to decrease the discrepancy measure between the current state's probability distribution and the final (diffused) state's probability distribution,
iv. the resulting sequence of probability distributions arising from the above transformations—the generated training data—are stored in the processor's associated memory,
v. the sequential transformation is conditioned on the target protein structure;
d. training a neural network, via the processor, to learn the constituent node locations of the ligand, wherein the neural network training process starts at the ligand's final diffused state, and sequentially recovers the preceding states, thereby ultimately yielding an approximation for the ligand's origin state:
i. wherein the training dataset is the generated training data,
ii. wherein for each voxel, the training loss includes a measure of discrepancy between the neural network's approximation of each state's probability distribution and the state's probability distribution in the training data,
iii. wherein the neural network is configured to accept a node composition as input, and to output the spatial position of the nodes of a corresponding ligand;
e. receiving, at a processor:
i. the node composition of a candidate ligand, and
ii. a target protein's sequence and structure; and
f. using the trained neural network to determine:
i. the spatial locations of the ligand's constituent nodes, and
ii. the ligand's docking site on the target protein.

7. The method of claim 6, wherein the final (diffused) state is such that for each voxel, the associated probability distribution is uniform over the ligand nodes, wherein each node of the ligand has equal probability of being in any given voxel.

8. The method of claim 7, wherein the discrepancy measure is a Kullback-Leibler divergence.

9. The method of claim 8, wherein the ligand is a peptide ligand, wherein the nodes represent the constituent amino acids.

10. A method, as in the method of claim 9, for obtaining the spatial locations of a peptide ligand's amino acids and its docking site on a target protein, given its amino acid composition and its target protein sequence and structure, wherein the method is also for obtaining the peptide ligand's structure, the method further comprising:
a. receiving, at a processor, the ligand's amino acid sequence;
b. obtaining, via the processor, the ligand's structure representation by:
i. using the method of claim 9 to obtain the spatial locations of the ligand's constituent amino acids,
ii. obtaining the adjacency information of the ligand's constituent amino acids from the ligand's amino acid sequence,
iii. obtaining the ligand's structure and docking coordinates from its amino acid spatial locations and its amino acid adjacency information.

11. The method of claim 8, wherein the ligand is a non-peptide drug ligand, wherein the non-peptide drug ligand is represented as a single node.

12. A method, as in the method of claim 9, for obtaining the spatial locations of a peptide ligand's amino acids and its docking site on a target protein, given its amino acid composition and its target protein sequence and structure, wherein the method is also for obtaining the peptide ligand's structure, the method further comprising:
a. training a ligand amino acid adjacency determining neural network conditioned on the target protein structure, wherein:
i. the training data include (i.1) spatial locations of ligand amino acids, and (i.2) associated target protein structure,
ii. training labels are the connections between the ligand amino acids;
b. using the node position locator neural network to determine the amino acid positions, and using the ligand amino acid adjacency determining neural network to determine the ligand amino acid connections;
c. obtaining a candidate ligand structure from its amino acid spatial positions and their connections.

13. A method, as in the method of claim 12, for obtaining a candidate peptide ligand's structure and docking site given only its amino acid composition and its target protein sequence and structure, wherein the method is also for obtaining a candidate peptide drug ligand for a given target protein, the method further comprising:
a. receiving, at a processor, a plurality of amino acid compositions;
b. for each amino acid composition in the plurality of compositions, using the method of claim 12 to obtain:
i. a candidate peptide drug structure, and
ii. the candidate peptide drug's docking site on the target protein;
c. based on docking site and structure, evaluating the interaction and efficacy of each candidate peptide drug ligand with the target protein;
d. selecting the most efficacious candidate peptide drug ligand from the plurality of candidate peptide drug ligands.

14. The method of claim 13, further comprising synthesizing the peptide drug ligand.

15. The method of claim 14, further comprising assessing the in vitro and in vivo biological activity of the peptide drug ligand in humans, animals, or plants.

16. A method, as in the method of claim 11, for obtaining the docking site of a non-peptide drug ligand on a target protein, wherein the method is also for obtaining a candidate non-peptide drug ligand for a given target protein, the method further comprising:
a. receiving, at a processor, a plurality of candidate non-peptide drug ligand embedding representations;
b. using the method of claim 11 to determine the docking site of each of the plurality of represented candidate non-polypeptide drug ligands on the target protein;
c. on a basis including docking site information, evaluating the interaction and efficacy of each represented candidate non-peptide drug ligand with the target protein;
d. selecting the most efficacious represented candidate non-peptide drug ligand from the plurality of represented candidate non-peptide drug ligands.

17. The method of claim 16, further comprising manufacturing the non-peptide drug ligand.

18. The method of claim 17, further comprising assessing the in vitro and in vivo biological activity of the non-peptide drug ligand in humans, animals, or plants.

19. An apparatus, comprising: a processor and an associated memory, wherein the memory stores instructions that when executed by the processor, cause the processor to:
a. receive representations of a plurality of protein-ligand complexes, wherein each ligand is a peptide ligand, and the representation of each target protein and of each ligand consists of:
i. an amino acid sequence, and
ii. an associated structure representation;
b. for each target protein and each associated ligand, represent its structure as voxel coordinates of its constituent amino acids within a three dimensional grid, wherein with each voxel is associated a probability distribution which indicates the probability of finding any given constituent amino acid in that voxel;
c. for each ligand, sequentially transform each voxel's probability distribution over the ligand amino acids, such that:
i. a measure of discrepancy between consecutive probability distributions is bounded by a hyperparameter function,
ii. a probability conservation constraint is respected, wherein for any given amino acid residue, the sum of the probabilities across voxels is constant,
iii. the sequential transformation proceeds in a manner to decrease the discrepancy measure between the current state's probability distribution and the final (diffused) state's probability distribution,
iv. the resulting sequence of probability distributions arising from the above transformations—the generated training data—are stored in the processor's associated memory,
v. the sequential transformation is conditioned on the target protein structure;
d. train a neural network to learn the constituent amino acid locations of the ligand, wherein the neural network training process starts at the ligand's final diffused state, and sequentially recovers the preceding states, thereby ultimately yielding an approximation for the ligand's origin state:
i. wherein the training dataset is the generated training data,
ii. wherein for each voxel, the training loss includes a measure of discrepancy between the neural network's approximation of each state's probability distribution and the state's probability distribution in the training data;
e. receive:
i. the amino acid composition of a candidate ligand, and
ii. a target protein's sequence and structure; and
f. use the trained neural network to determine:
i. the spatial locations of the ligand's constituent amino acids, and
ii. the ligand's docking site on the target protein.

20. The apparatus of claim 19, further comprising:
a. configuring the final (diffused) state such that for each voxel, the associated probability distribution is uniform over the ligand's amino acid residues, wherein each amino acid residue of the ligand has equal probability of being in any given voxel;
b. choosing as the discrepancy measure, a Kullback-Leibler divergence;
c. selecting a target protein of interest;
d. using the apparatus of claim 19 to determine the respective docking sites of a plurality of candidate peptide ligands on the target protein;
e. assessing the predicated efficacy of each of the plurality of candidate ligands based on their docking site, and assessing their properties based on their embedding location in a high dimensional protein space;
f. selecting the most effective of the candidate peptide ligands;
g. synthesizing the selected peptide ligand;
h. testing the biological activity of the synthesized peptide ligand in vitro and in vivo in humans, animals, or plants.
